(19) United States
(12) Patent Application Publication
(10) Pub. No.: US 2010/0284960 A1
(43) Pub. Date: Nov. 11, 2010

(54) Dosage Forms and Co-Administration of Opioid Agonist and Opioid Antagonist

(75) Inventor: Jennifer Riggs-Sauthier, Huntsville, AL (US)

Correspondence Address: JONES DAY, 222 EAST 41ST ST, NEW YORK, NY, 10017, US

(73) Assignee: Nektar Therapeutics AL, Corporation, Huntsville, AL (US)

(21) Appl. No.: 12/445,922

(22) PCT Filed: Nov. 7, 2007
(86) PCT No.: PCT/US07/23534

§ 371 Date: Jan. 19, 2010

Related U.S. Application Data
(60) Provisional application No. 60/857,610, filed on Nov. 7, 2006.

(51) Int. Cl. A61K 31/787; A61P 25/04; A61P 1/10; A61K 31/485

(52) U.S. Cl. 424/78.36; 514/282

(57) ABSTRACT

The present invention provides compositions comprising an opioid agonist and a water-soluble, non-peptidic polymer-opioid antagonist conjugate. Among other things, dosage forms and methods of administering the dosage forms are also provided.

### CROSS REFERENCE TO RELATED APPLICATION

This application claims the benefit of priority to provisional patent application Ser. No. 60/857,610, which is incorporated herein by reference.

### BACKGROUND OF THE INVENTION

The present invention relates generally to the co-administration of an opioid agonist and an opioid antagonist. In addition, the invention relates to, among other things, dosage forms for facile co-administration of an opioid agonist and an opioid antagonist, methods for administering an opioid agonist and an opioid antagonist, compositions containing an opioid agonist and an opioid antagonist, dosage forms containing a opioid agonist and an opioid antagonist, and so on.

### BACKGROUND OF THE INVENTION

In the treatment of a disease, a clinician will often prescribe a drug to be administered to her patient. In the ideal case, the drug will ameliorate the disease without causing substantial side effects. Unfortunately, very few drugs are associated with only very minor side effects.

Thus, in those circumstances where administration of a drug causes substantial side effects, the clinician must often prescribe a second drug to address the side effects caused by the first drug. Although doing so can satisfactorily address the side effect, such an approach introduces complexities for both the clinician and patient. For the clinician, concerns of drug interactions arise whenever two drugs for simultaneous (or near simultaneous) administration are indicated. For the patient, compliance concerns arise due to challenges introduced by having to coordinate the administration of two different drugs (potentially by different routes of administraton) and simply forgetting to take both drugs.

Pain treatment drugs (e.g., narcotic or opioid agonist drugs) are often associated with severe side effects that can be debilitating for patients. For example, the prevalence of constipation can be as high as 41% in patients receiving oral opioid therapy. Moore et al. (2005) Arthritis Research & Therapy 7:R1046-R1051. Opioid-induced constipation can lead to significant complications such that clinicians will only discharge those patients that return to normal bowel functioning following opioid therapy. The attendant costs associated with keeping these patients in the hospital has a dramatic and negative economic consequence.

The constipation-related and other side effects associated with opioid agonist drugs have been counteracted with the administration of opioid antagonist drugs. Ideally, the opioid antagonist drugs counteract the negative side effects (e.g., constipation),associated with administration of the opioid agonist, but without any substantial decrease in the treatment of pain. Alvimopan and methylnaltrexone have been suggested for use as opioid antagonists to counteract one or more side effects associated with opioid agonists. See, e.g., Yuan et al. (2006) Expert Opin Investig Drugs 15(5):541-552. Polymer conjugates of opioid antagonists have also been disclosed for use as opioid antagonists. See U.S. Patent Application Publication 2003/0124086. U.S. Patent Application Publication 2003/0124086 discloses that an opioid agonist and the polymer conjugate of an opioid antagonist can be administered in the same formulation.

There remains, however, a need for additional and/or specific formulations comprising a combination of a narcotic antagonist and narcotic agonist. The present invention addresses this and other needs in the art.

### SUMMARY OF THE INVENTION

In one or more embodiments of the invention, a composition is provided, the composition comprising a therapeutically effective amount of an opioid and a therapeutically effective amount of a polymer-opioid conjugate comprising a water-soluble, non-peptidic polymer covalently attached to an opioid antagonist, wherein the composition is preferably in the form selected from the group consisting of liquid, semi-solid, and solid. As used herein, a “polymer-opioid conjugate comprising a water-soluble, non-peptidic polymer covalently attached to an opioid antagonist” has the same meaning as a “polymer-opioid antagonist conjugate.”

In one or more embodiments of the invention, a unit dosage form is provided, the unit dosage form comprising a therapeutically effective amount of an opioid agonist and a therapeutically effective amount of a water-soluble, non-peptidic polymer-opioid antagonist conjugate.

In one or more embodiments of the invention, a method of administration is provided, the method comprising administering a composition comprising a therapeutically effective amount of an opioid and a therapeutically effective amount of a water-soluble, non-peptidic polymer-opioid antagonist conjugate, wherein the composition is preferably in the form selected from the group consisting of liquid, semisolid, and solid.

### DETAILED DESCRIPTION OF THE INVENTION

Before describing the present invention in detail, it is to be understood that this invention is not limited to the opioid agonists, polymer-opioid antagonist conjugates, and the like, as such may vary.

It must be noted that, as used in this specification and the claims, the singular forms “a,” “an,” and “the” include plural referents unless the context clearly dictates otherwise. Thus, for example, reference to a “polymer” includes a single polymer as well as two or more of the same or different polymers, reference to a “conjugate” refers to a single' conjugate as well as two or more of the same or different conjugates, reference to an “excipient” includes a single excipient as well as two or more of the same or different excipients, and the like.

In describing and claiming the present invention, the following terminology will be used in accordance with the definitions described below.

“PEG,” “polyethylene glycol” and “poly(ethylene glycol)” as used herein, are meant to encompass any water-soluble poly(ethylene oxide). Typically, PEGs for use in accordance with the invention comprise the following structure “—O(CH₂CH₂O)ₘ—” where (m) is 2 to 4000. As used herein, PEG also includes “—CH₂CH₂—O(CH₂CH₂O)ₘ—CH₂CH₂—” and “—(CH₂CH₂O)ₘ—,” depending upon whether or not the terminal oxygens have been displaced. When the PEG further comprises a spacer moiety (to be described in greater detail below), the atoms comprising the spacer moiety, when covalently attached to a water-soluble polymer segment, do not result in the formation of an oxygen-oxygen bond (i.e., an “—O—O—” or peroxide linkage). Throughout the specification and claims, it should be remembered that the term “PEG” includes structures having various terminal or “end capping” groups and so forth. The term “PEG” also means a polymer that contains a majority, that is to say, greater than 50%, of —CH₂CH₂O— monomeric subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” or “terminally capped” are interchangeably used herein to refer to a terminal or endpoint of a polymer having an end-capping moiety. Typically, although not necessarily, the end-capping moiety comprises a hydroxy or C₁₋₂₀ alkoxy group. Thus, examples of end-capping moieties include alkoxy (e.g., methoxy, ethoxy and benzyloxy), as well as aryl, heteroaryl, cyclo, heterocyclo, and the like. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polylner and/or the moiety (e.g., active agent) of interest to which the polymer is coupled to can be determined by using a suitable detector. Such labels include, without limitation, fluorescers, chemiluminescers, moieties used in enzyme labeling, colorimetric (e.g., dyes), metal ions, radioactive moieties, and the like, Suitable detectors include photometers, films, spectrometers, and the like.

“Non-naturally occurring” with respect to a polymer or water-soluble polymer means a polymer that in its entirety is not found in nature. A non-naturally occurring polymer or water-soluble polymer may, however, contain one or more subunits or portions of a subunit that are naturally occurring, so long as the overall polymer structure is not found in nature.

The term “water-soluble, non-peptidic polymer” is any polymer that is soluble in water at room temperature. Typically, a water-soluble, non-peptidic polymer will transmit at least about 75%, more preferably at least about 95% of light, transmitted by the same solution after filtering. On a weight basis, a water-soluble, non-peptidic polymer will preferably be at least about 35% (by weight) soluble in water, more preferably at least about 50% (by weight) soluble in water, still more preferably about 70% (by weight) soluble in water, and still more preferably about 85% (by weight) soluble in water. It is still more preferred, however, that the water-soluble, non-peptidic polymer is about 95% (by weight) soluble in water and most preferred that the water-soluble, non-peptidic polymer is completely soluble in water.

Molecular weight in the context of a water-soluble, non-peptidic polymer of the invention, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the invention are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

The term “reactive” or “activated” when used in conjunction with a particular functional group, refers to a reactive functional group that reacts readily with an electrophile or a nucleophile on another molecule. This is in contrast to those groups that require strong catalysts or highly impractical reaction conditions in order to react (i.e., a “nonreactive” or “inert” group).

As used herein, the term “functional group” or any synonym thereof is meant to encompass protected forms thereof.

The term “linkage” is used herein to refer to an atom or a collection of atoms used to link one moiety to another, such as a water-soluble polymer to an opioid antagonist. The linker is typically hydrolytically stable. In some instances, however, the linkage can include one or more physiologically hydrolyzable or enzymatically degradable linkages.

An “organic radical” as used herein includes, for example, alkyl, substituted alkyl, alkenyl, substituted alkenyl, alkynyl, substituted alkynyl, aryl and substituted aryl.

“Alkyl” refers to a hydrocarbon chain, typically ranging from about 1 to 20 atoms in length. Such hydrocarbon chain are preferably but not necessarily saturated and may be branched or straight chain, although typically straight chain is preferred. Exemplary alkyl groups include ethyl, propyl, butyl, pentyl, 1-methylbutyl, 1-ethylpropyl, 3-methylpentyl, and the like. As used herein, “alkyl” includes cycloalkyl when three or more carbon atoms are referenced and lower alkyl.

“Lower alkyl” refers to an alkyl group containing from 1 to 6 carbon atoms, and may be straight chain or branched, as exemplified by methyl, ethyl, n-butyl, iso-butyl, and tent-butyl.

“Cycloalkyl” refers to a saturated or unsaturated cyclic hydrocarbon chain, including bridged, fused, or, spiro cyclic compounds, preferably made up of 3 to about 12 carbon atoms, more preferably 3 to about 8.

“Non-interfering substituents” are those groups that, when present in a molecule, are typically non-reactive with other functional groups contained within the molecule.

The term “substituted” as in, for example, “substituted alkyl,” refers to a moiety (e.g., an alkyl group) substituted with one or more non-interfering substituents, such as, but not limited to: C₃-C₈ cycloalkyl, e.g., cyclopropyl, cyclobutyl, and the like; halo, e.g., fluoro, chloro, bromo, and iodo; cyano; alkoxy, lower phenyl (e.g., 0-2 substituted phenyl); substituted phenyl; and the like, for one or more hydrogen atoms. “Substituted aryl” is aryl having one or more non-interfering groups as a substituent. For substitutions on a phenyl ring, the substituents may be in any orientation (i.e., ortho, meta, or para). “Substituted ammonium” is ammonium having one or more non-interfering groups (e.g., an organic radical) as a substituent.

“Alkoxy” refers to an —O—R group, wherein R is alkyl or substituted alkyl, preferably C₁-C₂₀ alkyl (e.g., methoxy, ethoxy, propyloxy, benzyl, etc.), preferably C₁-C₇ alkyl.

As used herein, “alkenyl” refers to a branched or unbranched hydrocarbon group of 1 to 15 atoms in length, containing at least one double bond, such as ethenyl, n-propenyl, isopropenyl, n-butenyl, isobutenyl, octenyl, decenyl, tetradecenyl, and the like.

The term “alkynyl” as used herein refers to a branched or unbranched hydrocarbon group of 2 to 15 atoms in length, containing at least one triple bond, ethynyl, n-butynyl, isopentynyl, octynyl, decynyl, and so forth.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl. An aromatic moiety (e.g., Ar¹, Ar², and so forth), means a structure containing aryl.

“Heteroaryl” is an aryl group containing from one to four heteroatoms, preferably N, O, or S, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom which is not a carbon. Preferred heteroatoms include sulfur, oxygen, and nitrogen.

“Substituted heteroaryl” is heteroaryl having one or more non-interfering groups as substituents.

“Substituted heterocycle” is a heterocycle having one or more side chains formed from non-interfering substituents.

“Electrophile” refers to an ion or atom or collection of atoms, that may be ionic, having an electrophilic center, i.e., a center that is electron seeking, capable of reacting with a nucleophile.

“Nucleophile” refers to an ion or atom or collection of atoms that may be ionic having a nucleophilic center, i.e., a center that is seeking an electrophilic center or with an electrophile.

A “physiologically cleavable” or “hydrolyzable” bond is a relatively weak bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include, but are not limited to, carboxylate ester, phosphate ester, anhydrides, acetals, ketals, acyloxyalkyl ether, imines, ortho esters, peptides and oligonucleotides.

A “degradable linkage” includes, but is not limited to; a physiologically cleavable bond, a hydrolyzable bond, and an enzymatically degradable linkage. Thus, a “degradable linkage” is a linkage that may undergo either hydrolysis or cleavage by some other mechanism (e.g., enzyme-catalyzed, acid-catalyzed, base-catalyzed, and so forth) under physiological conditions. For example, a “degradable linkage” can involve an elimination reaction that has a base abstraction of a proton, (e.g., an ionizable hydrogen atom, H_{α}), as the driving force.

An “enzymatically degradable linkage” means a linkage that is subject to degradation by one or more enzymes.

A “hydrolytically stable” linkage or bond refers to a chemical bond, typically a covalent bond, that is substantially stable in water, that is to say, does not undergo hydrolysis under physiological conditions to, any appreciable extent over an extended period of time. Examples of hydrolytically stable linkages include but are not limited to the following: carbon-carbon bonds (e.g., in aliphatic chains), ethers, amides, urethanes (carbamates), and the like. Generally, a hydrolytically stable linkage is one that exhibits a rate of hydrolysis of less than about 1-2% per day under physiological conditions. Hydrolysis rates of representative chemical bonds can be found in most standard chemistry textbooks. It must be pointed out that some linkages can be hydrolytically stable or hydrolyzable, depending upon (for example) adjacent and neighboring atoms and ambient conditions. One of ordinary skill in the art can determine whether a given linkage or bond is hydrolytically stable or hydrolyzable in a given context by, for example, placing a linkage-containing molecule of interest under conditions of interest and testing for evidence of hydrolysis (e.g., the presence and amount of two molecules resulting from the cleavage of a single molecule). Other approaches known to those of ordinary skill in the art for determining whether a given linkage or bond is hydrolytically stable or hydrolyzable can also be used.

“Pharmaceutically acceptable excipient” or “pharmaceutically acceptable carrier” refers to an excipient that can be included in the compositions of the invention and that causes no significant adverse toxicological effects to the patient.

“Pharmacologically effective amount,” “physiologically effective amount,” and “therapeutically effective amount” are used interchangeably herein to mean the amount of an active agent (e.g., an opioid agonist, a polymer-opioid antagonist conjugate, and so forth) that is needed to provide a desired level of active agent in the bloodstream or in a target tissue. The exact amount will depend upon numerous factors, e.g., the particular active agent, the components and physical characteristics of the pharmaceutical preparation, intended patient population, patient considerations, and the like, and can readily be determined by one of ordinary skill in the art, based upon the information provided herein and available in the relevant literature.

“Multifunctional” in the context of a polymer of the invention means a polymer having 3 or more functional groups contained therein, where the functional groups may be the same or different. Multifunctional polymers of the invention will typically contain from about 3-100 functional groups, or from 3-50 functional groups, or from 3-25 functional groups, or from 3-15 functional groups, or from 3 to 10 functional groups, or will contain 3, 4, 5, 6, 7, 8, 9 or 10 functional groups within the polymer. A “difunctional” polymer means a polymer having two functional groups contained therein, either the same (i.e., homodifunctional) or different (i.e., heterodifunctional).

“Branched,” in reference to the geometry or overall structure of a polymer, refers to polymer having 2 or more polymer “arms.” A branched polymer may possess 2 polymer arms, 3 polymer arms, 4 polymer arms, 6 polymer arms, 8 polymer arms or more. One particular type of highly branched polymer is a dendritic polymer or dendrimer, which, for the purposes of the invention, is considered to possess a structure distinct from that of a branched polymer.

A “dendrimer” or dendritic polymer is a globular, size monodisperse polymer in which all bonds emerge radially from a central focal point or core with a regular branching pattern and with repeat units that each contribute a branch point. Dendrimers exhibit certain dendritic state properties such as core encapsulation; making them unique from other types of polymers.

A basic or acidic reactant described herein includes neutral, charged, and any corresponding salt forms thereof.

The term “patient,” refers to a living organism suffering from or prone to a condition that can be prevented or treated by administration of a conjugate as provided herein,' and includes both humans and animals.

“Optional” and “optionally” mean that the subsequently described circumstance may or may not occur, so that the description includes instances where the circumstance occurs and instances where it does not.

As used herein, the “halo” designator (e.g., fluoro, chloro, iodo, bromo, and so forth) is generally used when the halogen is attached to a molecule, while the suffix “ide” (e.g., fluoride, chloride, iodide, bromide, and so forth) is used when the halogen exists in, its independent ionic form (e.g., such as when a leaving group leaves a molecule).

In the context of the present discussion, it should be recognized that the definition of a variable provided with respect to one structure or formula is applicable to the same variable repeated in a different structure, unless the context dictates otherwise.

As previously stated, the present invention comprises (among other things) a composition comprising a therapeutically effective amount of an opioid agonist and a therapeutically effective amount of a polymer-opioid antagonist conjugate comprising a polymer covalently attached to an opioid antagonist, wherein the composition is in the form selected from the group consisting of liquid, semi-solid, and solid.

Opioid Agonists

As used herein, an “opioid agonist” is any natural or synthetic alkaloid or structural derivative of opium that activates one or more opioid receptor types, including partial agonists (i.e., compounds exhibiting activity against less than all opioid receptor types) and agonist-antagonists (i.e., compounds exhibiting agonist activity at one receptor type and antagonist activity at another receptor type). The opioid agonist can be a natural alkaloid such as a penanthrene (e.g., morphine) or benzylisoquinoline (e.g., papaverine), a semi-synthetic derivative (e.g., hydromorphone), or any of various classes of synthetic derivatives (e.g., phenylpiperidines, benzmorphans, priopionanilides, and morphinans). Exemplary opioid agonists include 1-a-acetylmethadol, alfentanil, alphaprodine, anileridine, bremazocine, buprenorphine, butorphanol, codeine, cyclazocine, dezocine, diacetylmorphine (i.e., heroin), dihydrocodeine, ethylmorphine, fentanyl, hydrocodorie, hydromorphone, levorphanol, meperidine (i.e., pethidine), methadone, methotrimeprazine, morphine, nalbuphine, nefopam, normophine, noscapine, oxycodone, oxymorphone, papaverine, pentazocine, pethidine, phenazocine, propiram, propoxyphene, sufentanil, thebaine and tramadol, and, pharmaceutically acceptable salts of each of the foregoing. Structures of preferred opioid agonists are provided below:

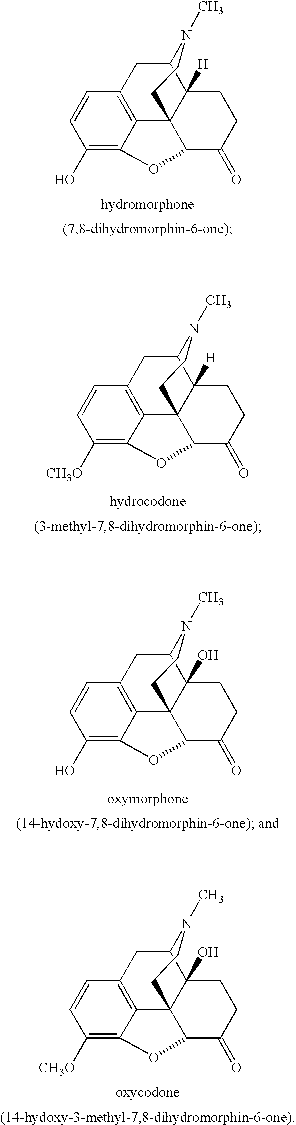

The Polymer-Opioid Antagonist Conjugate

The polymer-opioid antagonist conjugate comprises a water-soluble and non-peptidic polymer covalently attached (either directly or through one or more atoms) to an opioid antagonist. The polymer-opioid antagonist conjugate typically comprises a polymer having a molecular weight selected such that the conjugate does not pass to any appreciable degree through the blood-brain barrier and into the central nervous system.

Suitable polymers for covalent attachment to an opioid antagonist include poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), poly(acrylic acid), carboxymethylcellulose, hyaluronic acid, hydroxypropylmethyl cellulose, and copolymers, terpolymers, and mixtures thereof. A preferred polymer is a polyethylene glycol.

The polymer may be linear, branched, or forked. With respect to a linear polymer, the conjugate may incorporate a heterobifunctional or a homobifunctional polymer. A conjugate of a heterobifunctional polymer is one wherein one terminus of the polymer attached to the opioid antagonist and the other terminus is functionalized with a different moiety. A′ conjugate of a′ homobifunctional polymer possesses a structure wherein each end of a linear polymer is covalently attached to an opioid antagonist, typically by an identical linkage.

Typically, the number average molecular weight of the polymer of the polymer-opioid antagonist conjugate is less than about 5,000 daltons (Da), and more preferably is less than about 2,000 Da. An exemplary number average molecular weight for the will fall within one or more of the following ranges: from about 100 Da to, about 2,000 Da; from about 100 Da to about 1,800 Da; from about 100 Da to about 1,600 Da; from about 100 Da to about 1,500 Da; from about 100 Da to about 1,200 Da; from about 100 Da to about 1,000 Da; from about 100 Da to about 800 Da; from about 100 Da to about 500 Da; from about 300 Da to about 2,000 Da; and from about 300 Da to about 1,000 Da. Polymers having a number average molecular weight of about 100 Da, about 200 Da, about 300 Da, about 400 Da, about 500 Da, about 550 Da, about 600 Da, about 700 Da, about 800 Da, about 900 Da and about 1,000 Da are particularly preferred. The polymers of the invention are hydrophilic in nature.

The linkage between the polymer and the opioid antagonist is preferably hydrolytically stable so that the opioid antagonist is not released from the polymer following administration to a patient. Release of the opioid antagonist in vivo could lead to a loss in analgesic effect of the opioid compound due to passage of the released opioid antagonist into the central nervous system. Representative linkages for connecting the opioid antagonist and the polymer include ether, amide, urethane (also known as carbamate), amine, thioether (also known as sulfide), and urea (also known as carbamide) linkages. In some instances, however, a degradable linkage or a hydrolyzable linkage between the polymer and the opioid antagonist.

The particular linkage and linkage chemistry employed will depend upon the opioid antagonist, functional groups within the molecule available either for attachment to a polymer or conversion to a suitable attachment site, the presence of additional functional groups within the molecule, and the like, and can be readily determined by one skilled in the art based upon the guidance presented herein.

The polymer-opioid antagonist conjugate maintains at least a measurable degree of specific opioid antagonist activity. That is to say, the polymer-opioid antagonist conjugate possesses anywhere from about 1% to about 100% or more of the specific activity of the unmodified parent opioid antagonist compound. Such activity may be determined using a suitable in-vivo or in-vitro model, depending upon the known activity of the particular opioid antagonist parent compound.

For example, a hot plate or tail flick analgesia assay can be used to assess the level of antagonist activity of the polymer conjugates of the invention (See, for example, Tulunay et al. (1974) J. Pharmacol Exp Ther 190:395-400; Takahashi et al. (1987) Gen Pharmacol 18(2):201-3; and Fishman et al. (1975) Pharmacology 13(6):513-9. In general, a polymer conjugate will possess a specific activity of at least about 2%, 5%, 10%, 15%, 25%, 30%, 40%, 50%, 60%, 70%, 80%, 90% or more relative to that of the unmodified parent opioid antagonist, when measured in a suitable model, such as those well known in the art. Preferably, a conjugate will maintain at least 50% or more of the opioid antagonist activity of the unmodified parent compound.

The opioid antagonist used in the formation of the polymer-opioid antagonist conjugate is any opioid antagonist that can be conjugated to a polymer. Preferred opioid antagonists are based on the structure of a morphinone. The morphinone is a phenanthrene-based moiety that (a) comprises the following structure:

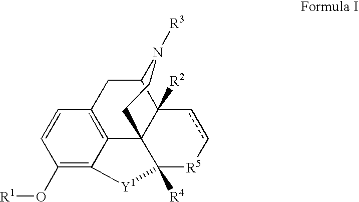

wherein:

R¹ is H or an organic radical;

R² is H or OH;

R³ is H or an organic radical; (preferably R³ is H or an organic radical with the proviso that When R³ is an organic radical, the organic radical is not

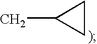

R⁴ is H or an organic radical;

the dotted line (“- - - ”) represents an optional double bond;

Y¹ is O or S; and

R⁵ is selected from the group consisting of

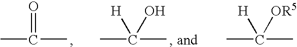

(without regard to stereochemistry), wherein R⁶ is an organic radical.

It is particularly preferred that the morphinone comprises the following structure:

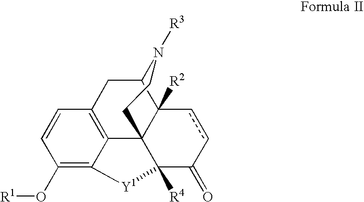

### Wherein:

R¹ is H or an organic radical;

R² is H or OH;

R³ is H or an organic radical, with the proviso that when R³ is an organic radical, the organic radical is not

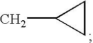

R⁴ is H or an organic radical;

the dotted line (“- - - ”) represents an optional double bond; and

Y¹ is O or S.

Exemplary morphinones upon which an opioid antagonist can be derived include: hydromorphone; hydrocodone; oxymorphone; oxycodone;

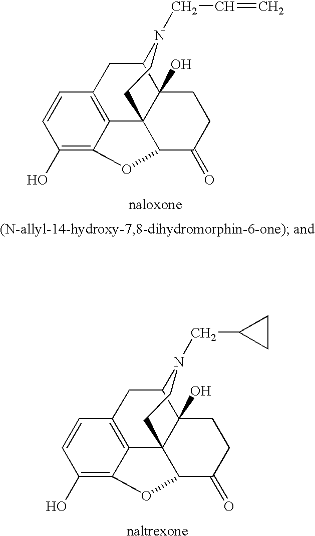

### (N-cyclopropylmethyl-14-hydroxy-7,8-dihydromorphin-6-one)

Whether any given structure can serve as an opioid antagonist can be determined by one of ordinary skill in the art.

These and other morphinones have been described and characterized previously. See, for example: U.S. Pat. Nos. 2,628,962, 2,654,756 and 2,649,454 (hydromorphone and others); U.S. Pat. No. 2,715,626 (hydrocodone and others); U.S. Pat. No. 2,806,033 (oxymorphone and others); Freund et al. (1916) J. Prak. Chemie 94:135-178 (oxycodone); U.S. Pat. No. 3,254,088 (naloxone and others); and U.S. Pat. No. 3,332,950 (naltrexone and others).

The polymer conjugate of the invention can be formed using known techniques for covalent attachment of in activated polymer, such as an activated PEG, to a biologically active agent (See, for example, POLY(ETHYLENE GLYCOL) CHEMISTRY AND BIOLOGICAL APPLICATIONS, American Chemical Society, Washington, D.C. (1997)). The general method involves selection of a reactive polymer bearing a functional group suitable for reaction with a functional group of the opioid antagonist molecule and reaction of the reactive polymer with the opioid antagonist in solution to form a covalently-bound conjugate.

Selection of the functional group of the polymer will depend, in, part, on the functional group on the opioid antagonist molecule. The functional group of the polymer is preferably chosen to result in formation of a hydrolytically stable linkage between the opioid antagonist and the polymer. A polymer of the invention suitable for coupling to an opioid antagonist molecule will typically have a terminal functional group such as the following: N-succinimidyl carbonate (see e.g., U.S. Pat. Nos. 5,281,698, 5,468,478), amine (see, e.g., Buckmann et al. Makromol. Chem. 182:1379 (1981), Zalipsky et al. Eur. Polym. 3. 19:1177 (1983)), hydrazide (See, e.g., Andresz et al. Makromol. Chem. 179:301 (1978)), succinimidyl propionate and succinimidyl butanoate (see, e.g., Olson et al. in Poly(ethylene glycol) Chemistry & Biological Applications, pp 170-181, Harris & Zalipsky Eds., ACS, Washington, D.C., 1997; see also U.S. Pat. No. 5,672,662), succinimidyl succinate (See, e.g., Abuchowski et al. Cancer Biochem. Biophys. 7:175 (1984) and Joppich et al., Makromol. Chem. 180:1381 (1979), succinimidyl ester (see, e.g., U.S. Pat. No. 4,670,417), benzotriazole carbonate (see, e.g., U.S. Pat. No. 5,650,234), glycidyl ether (see, e.g., Pitha et al. Eur. J. Biochem. 94:11 (1979), Elling et al., Biotech. Appl. Biochem. 13:354 (1991), oxycarbonylimidazole (see, e.g., Beauchamp, et al., Anal. Biochem. 131:25 (1983), Tondelli et al. J. Controlled Release 1:251 (1985)), p-nitrophenyl carbonate (see, e.g., Veronese, et al., Appl. Biochem. Biotech., 11:141 (1985); and Sartore et al., Appl. Biochem. Biotech., 27:45 (1991)), aldehyde (see, e.g., Harris et al. J. Polym. Sci. Chem. Ed. 22:341 (1984), U.S. Pat. No. 5,824,784, U.S. Pat. No. 5,252,714), maleimide (see, e.g., Goodson et al. Bio/Technology 8:343 (1990), Romani et al. in Chemistry of Peptides and Proteins 2:29 (1984)), and Kogan, Synthetic Comm. 22:2417 (1992)), orthopyridyl-disulfide (see, e.g., Woghiren, et al. Bioconj. Chem. 4:314 (1993)), acrylol (see, e.g., Sawhney et al., Macromolecules, 26:581 (1993)), vinylsulfone (see, e.g., U.S. Pat. No. 5,900,461). All of the above references are incorporated herein by reference.

In one embodiment, the polymer-opioid antagonist conjugate will have the following structure:

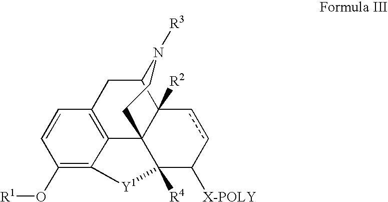

wherein:

R¹ is H or an organic radical;

R² is H or OH;

R³ is H or an organic radical, (preferably R³ is H or an organic radical such as C₁₋₆ alkyl, substituted C₁₋₆ alkyl, C₃₋₆ cycloalkyl, substituted C₃₋₆ cycloalkyl, C₂₋₆ alkenyl, substituted C₂₋₆ alkenyl, C₂₋₆ alkynyl; substituted C₂₋₆ alkynyl, aryl, substituted aryl, heteroaryl, substituted heteroaryl, heterocycle, and substituted heterocycle, with the proviso that when R³ is an organic radical, the organic radical is not

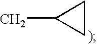

R⁴ is H or an organic radical;

the dotted line (“- - - ”) represents an optional double bond;

Y¹ is O or S;

X is a linkage, preferably a hydrolytically stable linkage covalently attaching the polymer to the rest of the molecule; and

POLY is a residue of a water-soluble and non-peptidic polymer.

In one embodiment; the polymer-opioid antagonist conjugate will have the following structure:

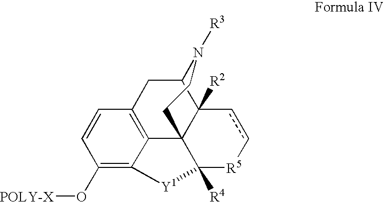

wherein:

R¹ is H or an organic radical;

R² is H or OH;

R³ is H or an organic radical, (preferably R³ is H or an organic radical such as C₁₋₆ alkyl, substituted C₁₋₆ alkyl, C₃₋₆ cycloalkyl, substituted C₃₋₆ cycloalkyl; C₂₋₆ alkenyl, substituted C₂₋₆ alkenyl, C₂₋₆ alkynyl, substituted C₂₋₆ alkynyl, aryl, substituted aryl, heteroaryl, substituted heteroaryl, heterocycle, and substituted heterocycle with the proviso that when R³ is an organic radical, the organic radical is not

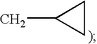

R⁴ is H or an organic radical;

the dotted line (“- - - ”) represents an optional double bond;

Y¹ is O or S;

R⁵ is selected from the group consisting of

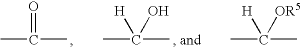

(without regard to stereochemistry), wherein R⁶ is an organic radical; and

X is a linkage, preferably a hydrolytically stable linkage covalently attaching the polymer to the rest of the molecule; and

POLY is a residue of a water-soluble and non-peptidic polymer.

In one embodiment, the polymer-opioid antagonist conjugate will have the following structure

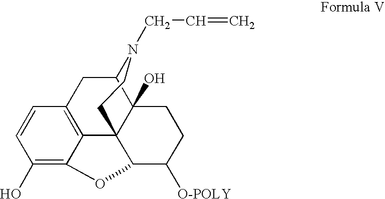

wherein POLY is a water-soluble polymer, preferably —(CH₂CH₂O)ₙ—CH₃ (wherein “n” is an integer from 3 to 14, preferably about 5 to 9.

Examples of the above-described conjugates can be found in U.S. Patent Application Publication No. 2003/0124086 and U.S. Patent Application Publication No. 2005/0136031.

Dosage Forms

Depending on the intended mode of administration, the composition may be a liquid, semi-solid or solid. Exemplary liquids include a suspension, a solution, an emulsion, and a syrup, which can be formulated for administration to a patient. Exemplary semi-solids include gels which can be administered “as is” or formulated (e.g., into a gel-cap) for administration to a patient. Exemplary solids include granules, pellets, beads, powders, which can be administered “as is” or formulated into one or more of the following for administration to a patient: a tablet; a capsule; a caplet; a suppository; and a troche. Preferably, the composition will be in a unit dosage form to thereby provide a unit dosage suitable for single administration of a dosage of each active component in the unit dosage form. Suitable pharmaceutical compositions and dosage forms may be prepared using conventional methods known to those in the field of pharmaceutical formulation and described in the pertinent texts and literature, e.g., in Remington's Pharmaceutical Sciences: 18^{th} Edition, Gennaro, A. R., Ed. (Mack Publishing Company; Easton, Pa.; 1990).

Oral dosage forms are preferred and include tablets, capsules, caplets, gel caps, troches, solutions, suspensions, and syrups. Tablets and capsules represent the most convenient oral dosage forms.

Tablets can be manufactured using standard tablet processing procedures and equipment. Preferred techniques for forming tablets include direct compression and granulation. In addition to the active agents, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc; kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

In some instances, the tablet can be in the form of a uniform tablet. In uniform tablets, the formulation used in preparing the tablet is a substantially homogenous mixture of active agents and one or more pharmaceutical excipient (e.g., diluent). The formulation is then used to make tablets using a suitable tableting process to thereby result in a tablet that is substantially homogenous throughout the tablet.

In still other instances, the tablet can also take the form of a layered tablet (of one, two, three or more layers). The method for manufacturing the layered tablet can include combining two different formulations (e.g., one formulation containing the opioid agonist and another containing the polymer-opioid conjugate) and compressing the two together to form the tablet. Multiple layered tablets of three or more layers are also possible and can be formed, for example, in a similar manner by combining three or more distinct formulations and followed by compression.

Optionally, a barrier layer can be included in the layered tablet. One approach for incorporating a barrier layers involves forming a compressed first layer of a first formulation (e.g., a formulation containing a first active agent) wherein the compress layers has one exposed surface, coating the exposed surface with a material (e.g., a material that is substantially impermeable to thereby prevent physical interaction between adjacent layers) to form a coated surface, and contacting the coated surface with a second formulation (e.g., a second formulation containing a second active agent), and compressing the second formulation and coated surface to form a layered tablet having a barrier layer included therein.

Capsules are also preferred oral dosage forms, in which case the composition may be encapsulated in the form of a liquid, semi-solid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington's Pharmaceutical Sciences, supra, which describes materials and methods for preparing encapsulated pharmaceuticals.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The pharmaceutical preparations encompass all types of formulations. The amount of the active agents (i.e., opioid agonist and the polymer-opioid antagonist conjugate) in the composition will vary depending on a number of factors, but will optimally be a therapeutically effective dose of each active agent when the composition is stored in a unit dose form. A therapeutically effective dose for each active agent can be determined experimentally by repeated administration of increasing amounts of the active agent in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 2%-98% (should this be 2%-98%?) by weight, more preferably from about 5-95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The invention also provides a method for administering a composition as provided herein to a patient suffering from a condition that is responsive to treatment with an opioid agonist. Preferrably, this method comprises administering a unit dosage form as described herein. The method of administering may be used to treat any condition that can be remedied or prevented by administration of the opioid agonist (e.g., moderate to severe pain). Those of ordinary skill in the art appreciate which conditions an opioid agonist can effectively treat. The actual dose to be administered will vary depend upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a therapeutically effective amount will range from about 0.001 mg to 100 mg, preferably in doses from 0.01 mg/day to 75 mg/day, and more preferably in doses from 0.10 mg/day to 50 mg/day.

Exemplary therapeutically effective amounts of the water-soluble, non-peptidic polymer-opioid antagonist (which can be present in a single unit dosage form) include: an amount from 5 mg to 250 mg; 5 mg; 25 mg; 50 mg; and 100 mg. Exemplary therapeutically effective amounts of opioid agonists (which can be present in a single unit dosage form) include: 30 mg to 450 mg of morphine; 200 mg to 3,000 mg of codeine; 5 mg to 450 mg of hydrocodone; 7 mg to 112 mg of hydromorphone; 20 mg to 300 mg of oxycodone; and 10 mg to 150 mg of oxymorphone.

In some instances, the unit dosage form contains: from 0.8 mg to 17 mg of the water-soluble, non-peptidic polymer-opioid antagonist and 5 mg to 65 mg of morphine (intended to be taken, for example, every four hours); from 0.8 mg to 17 mg of the water-soluble, non-peptidic polymer-opioid antagonist and 33 mg to 500 mg of codeine (intended to be taken, for example, every four hours); from 0.8 mg to 17 mg of the Water-soluble, non-peptidic polymer-opioid antagonist and 5 mg to 65 mg of hydrocodone (intended to be taken, for example, every four hours); from 0.8 mg to 17 mg of the water-soluble, non-peptidic polymer-opioid antagonist and 1.2 mg to 19 mg of hydromorphone (intended to be taken, for example, every four hours); and from 0.8 mg to 17 mg of the water-soluble, non-peptidic polymer-opioid antagonist and 3 mg to 50 mg of oxycodone (intended to be taken, for example, every four hours).

The unit dosage form can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

It is to be understood that while the invention has been described in conjunction with the preferred specific embodiments thereof, that the foregoing description as well as the experimental that follow are intended to illustrate and not limit the scope of the invention.

Other aspects, advantages and modifications within the scope of the invention will be apparent to those skilled in the art to which the invention pertains.

All articles, books, patents, patent publications and other publications referenced herein are hereby incorporated by reference in their entireties.

### EXPERIMENTAL

The practice of the invention will employ, unless otherwise indicated, conventional techniques of organic synthesis and the like, which are understood by one of ordinary skill in the art and are explained in the literature. In the following examples, efforts have been made to ensure accuracy with respect to numbers used (e.g., amounts, temperatures, and so forth), but some experimental error and deviation should be accounted for. Unless otherwise indicated, temperature is in degrees Celsius and pressure is at or near atmospheric pressure at sea level. All reagents were obtained commercially unless otherwise indicated. All generated NMR was obtained from a 300 or 400 MHz NMR spectrometer manufactured by Bruker (Billerica, Mass.). All processing is carried out in glass or glass-lined vessels and contact with metal-containing vessels or equipment is avoided.

An exemplary water-soluble, non-peptidic polymer-opioid antagonist conjugate (the “polymer-opioid antagonist conjugate”) having the structure of Formula V, wherein POLY is —(CH₂CH₂O)₇—CH₃ (the “polymer-opioid antagonist conjugate”) is used in the following examples.

### Example 1

Oral morphine sulfate 10 mg/5 mL solution (100 mL) and an amount of polymer-opioid antagonist conjugate sufficient to provide 25 mg/5 mL of the polymer-opioid antagonist conjugate in the resulting liquid are combined followed by stirring to form a liquid composition. A unit dosage form is prepared by placing 5 mL of the composition in an oral syringe.

### Example 2

Oral morphine sulfate 20 mg/5 mL solution (100 mL) and an amount of polymer-opioid antagonist conjugate sufficient to provide 25 mg/5 mL of the polymer-opioid antagonist conjugate in the resulting liquid are combined followed by stirring to form a liquid composition. A unit dosage form is prepared by placing 5 mL of the composition in an oral syringe.

### Example 3

Oxycodone HCl 5 mg and acetaminophen 325 mg/5 mL solution (Roxicet™ solution, Roxane Laboratories, Columbus Ohio) and an amount of polymer-opioid antagonist conjugate sufficient to provide 50 mg/5 mL of the polymer-opioid antagonist conjugate in the resulting liquid are combined followed by stirring to form a liquid composition. A unit dosage form is prepared by placing 5 mL of the composition in an oral syringe.

### Example 4

Hydrocodone 1 mg/5 mL (Sigma, St. Louis, Mo.) and an amount of polymer-opioid antagonist conjugate sufficient to provide 5 mg/5 mL of the polymer-opioid antagonist conjugate in the resulting liquid are combined followed by stirring to form a liquid composition. A unit dosage form is prepared by placing 20 mL of the composition in an oral syringe.

### Example 5

A 30 mg codeine sulfate tablet is ground into a powder and combined with 25 mg of the polymer-opioid antagonist conjugate. Lactose in an amount sufficient to fill a capsule size conventionally referred to as “1” is added and thoroughly mixed until a uniform powder is formed. A unit dosage form is prepared by housing the uniform powder into a capsule.

### Example 6

Hydrocodone (10 mg) in powder form is combined with 25 mg of the polymer-opioid antagonist conjugate. Lactose in an amount sufficient to fill a capsule size conventionally referred to as “1” is added and thoroughly mixed until a uniform powder is formed. A unit dosage form is prepared by housing the uniform powder into a capsule.

### Example 7

Hydrocodone (5 mg) in powder form is combined with 50 mg of the polymer-opioid antagonist conjugate. Lactose in an amount sufficient to fill a capsule size conventionally referred to as “1” is added and thoroughly mixed until a uniform powder is formed. A unit dosage form is prepared by housing the uniform powder into a capsule.

### Example 8

Oxycodone HCl (5 mg) in powder form is combined with 25 mg of the polymer-opioid antagonist conjugate. Lactose in an amount sufficient to fill a capsule size conventionally referred to as “1” is added and thoroughly mixed until a uniform powder is formed. A unit dosage form is prepared by housing the uniform powder into a capsule.

### Example 9

Oxycedone HCl (10 mg) in powder form is combined with 25 mg of the polymer-opioid antagonist conjugate. Lactose in an amount sufficient to fill a capsule size conventionally referred to as “1” is added and thoroughly mixed until a uniform powder is formed. A unit dosage form is prepared by housing the uniform powder into a capsule.

### Example 10

Morphine sulfate (30 mg) in powder form is combined with 25 mg of the polymer-opioid antagonist conjugate. Lactose in an amount sufficient to fill a capsule size conventionally referred to as “1” is added and thoroughly mixed until a uniform powder is formed. A unit dosage form is prepared by housing the uniform powder into a capsule.

What is claimed is:

1. A composition comprising a therapeutically effective amount of an opioid agonist and a therapeutically effective amount of a water-soluble, non-peptidic polymer-opioid antagonist conjugate.

2. The composition of claim 1, wherein the opioid antagonist is selected from the group consisting of buprenorphine, cyclazocine, cyclorphan, naloxone, 6-aminonaloxone, N-methylnaloxone, naltrexone, 6-amino-naltrexone, N-methylnaltrexone, nalmephene, levallorphan, nalbuphine, naltrendol, naltrindole, nalorphine, norbinaltorphimine, oxilorphan, pentazocine, piperidine-N-alkylcarboxylate opioid antagonists, and opioid antagonist polypeptides.

3. The composition of claim 1, wherein the polymer-opioid antagonist conjugate comprises a hydrolytically stable linkage links the water-soluble, non-peptidic polymer covalently and the opioid antagonist.

4. The composition of claim 3, wherein the hydrolytically stable linkage is selected from the group consisting of amide, amine, carbamate, sulfide, ether, thioether, and urea.

5. The composition of claim 3, wherein the hydrolytically stable linkage is ether.

6. The composition of claim 1, wherein the molecular weight of the polymer is less than about 2,000 Da.

7. The composition of claim 1, wherein the opioid agonist is selected from the group consisting of alfentanil, bremazocine, buprenorphine, butorphanol, codeine, cyclazocine, dezocine, diacetylmorphine, dihydrocodeine, fentanyl, hydrocodone, hydromorphone, levorphanol, meperidine, methadone, morphine, nalbuphine, noscapine, oxycodone, oxymorphone, papaverine, pentazocine, pethidine, phenazocine, propiram, propoxyphene, sufentanil, thebaine and tramadol.

8. The composition of claim 7, wherein the opioid agonist is selected from the group consisting of oxycodone, hydrocodone and morphine.

9. The composition of claim 1, wherein the polymer-opioid antagonist conjugate has the following structure: 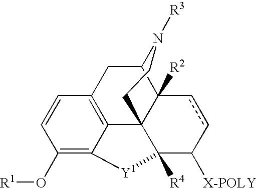
wherein:
R¹ is H or an organic radical;
R² is H or OH;
R³ is H or an organic radical;
R⁴ is H or an organic radical;
the dotted line (“- - - ”) represents an optional double bond;
Y¹ is O or S;
X is a linkage; and
POLY is a residue of a water-soluble, non-peptidic polymer.

10. The composition of claim 9, wherein the polymer-opioid antagonist conjugate has the following structure: 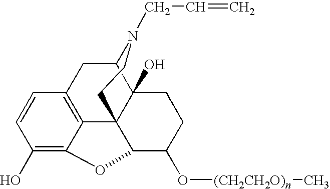
wherein “n” is an integer from 3 to 14.

11. The composition of claim 1, in solid form.

12. The composition of claim 1, in semi-solid form.

13. The composition of claim 1, in liquid form.

14. A unit dosage form comprising a therapeutically effective amount of an opioid agonist and a therapeutically effective amount of a water-soluble, non-peptidic polymer-opioid antagonist conjugate.

15. The unit dosage form of claim 14, wherein the opioid antagonist is selected from the group consisting of buprenorphine, cyclazocine, cyclorphan, naloxone, 6-amino-naloxone, N-methylnaloxone, naltrexone, 6-amino-naltrexone, N-methylnaltrexone, nalmephene, levallorphan, nalbuphine, naltrendol, naltrindole, nalorphine, nor-binaltorphimine, oxilorphan, pentazocine, piperidine-N-alkylcarboxylate opioid antagonists, and opioid antagonist polypeptides.

16. The unit dosage form of claim 14, wherein the polymer-opioid antagonist conjugate comprises a hydrolytically stable linkage links the water-soluble, non-peptidic polymer covalently and the opioid antagonist.

17. The unit dosage form of claim 16, wherein the hydrolytically stable linkage is selected from the group consisting of amide, amine, carbamate, sulfide, ether, thioether, and urea.

18. The unit dosage form of claim 16, wherein the hydrolytically stable linkage is ether.

19. The unit dosage form of claim 14, wherein the molecular weight of the polymer is less than about 2,000 Da.

20. The unit dosage form of claim 14, wherein the opioid agonist is selected from the group consisting of alfentanil, bremazocine, buprenorphine, butorphanol, codeine, cyclazocine, dezocine, diacetylmorphine, dihydrocodeine, fentanyl, hydrocodone, hydromorphone, levorphanol, meperidine, methadone, morphine, nalbuphine, noscapine, oxycodone, oxymorphone, papaverine, pentazocine, pethidine, phenazocine, propiram, propoxyphene, sufentanil, thebaine and tramadol.

21. The unit dosage form of claim 20, wherein the opioid agonist is selected from the group consisting of oxycodone, hydrocodone and morphine.

22. The unit dosage form of claim 14, wherein the polymer-opioid antagonist conjugate has the following structure: 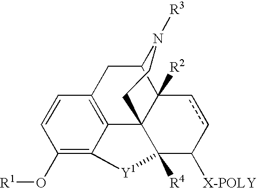
wherein:
R¹ is H or an organic radical;
R² is H or OH;
R³ is H or an organic radical;
R⁴ is H or an organic radical;
the dotted line (“- - - ”) represents an optional double bond;
Y¹ is O or S;
X is a linkage; and
POLY is a residue of a water-soluble, non-peptidic polymer.

23. The unit dosage form of claim 22, wherein the polymer-opioid antagonist conjugate has the following structure: 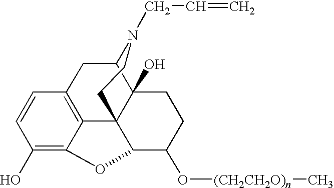
wherein “n” is an integer from 3 to 14.

24. A method comprising administering a composition comprising a therapeutically effective amount of an opioid agonist and a therapeutically effective amount of a water-soluble, non-peptidic polymer-opioid antagonist conjugate.

25. The method of claim 24, wherein the composition is administered orally.
